(19) United States
(12) Patent Application Publication
(10) Pub. No.: US 2005/0192544 A1
(43) Pub. Date: Sep. 1, 2005

(54) Syringe, in particular for medical applications

(76) Inventors: Peter Wolbring, St. Wendel (DE); Sascha Koeppel, Horn (CH)

Correspondence Address: HARNESS, DICKEY & PIERCE, P.L.C., P.O. BOX 828, BLOOMFIELD HILLS, MI, 48303, US

(21) Appl. No.: 11/062,349

(22) Filed: Feb. 18, 2005

(30) Foreign Application Priority Data
Feb. 20, 2004 (DE) 10 2004 009 919.7

(51) Int. Cl. A61M 5/315

(52) U.S. Cl. 604/218; 604/227

(57) ABSTRACT

A syringe has a syringe barrel comprising a proximal end and a distal end. At the proximal end there is a backstop with which a plunger stopper arranged in the syringe barrel is secured against inadvertent withdrawal. The backstop has at least two claw-like catch hooks arranged outside the syringe barrel at mutually offset circumferential positions. According to one aspect of the invention, the catch hooks are arranged on a plate-shaped finger support sitting at the proximal end of the syringe barrel.

### BACKGROUND OF THE INVENTION

The present invention relates to a syringe and more particularly to a syringe for medical applications. The invention further relates to a backstop for such a syringe in order to prevent inadvertent removal of a plunger stopper from the syringe.

In the pharmaceutical industry, there are a number of medicaments which, under sterile conditions, are introduced by the pharmaceutical manufacturer into a syringe barrel closed at the distal end with a sealing cap. The syringe barrel is then closed at the proximal end with a sterile plunger stopper, often made of rubber elastic material. The plunger rod of the syringe is then usually screwed into an internal thread on the plunger stopper, the syringe is packed in a sterile manner, and made ready for dispatch. A particular advantage of this procedure is that the medicament is in this way kept sterile until its actual use, something which is not always guaranteed when, for example, the medicament is drawn into the syringe just before being administered.

It is clear that this sterility is no longer afforded if the plunger stopper is inadvertently withdrawn from the syringe barrel during or before administration of the medicament. In the case of very expensive medicaments, the financial loss is correspondingly high. If, in addition, the medicament is highly toxic, for example an anti-cancer agent, inadvertent withdrawal of the plunger stopper also poses a hazard to the physician and/or patient.

The inadvertent withdrawal of the plunger stopper from the syringe barrel can be prevented by a backstop (in some cases also called a plunger brake). A large number of such backstops are known in the prior art. For example, WO 94/13339 discloses a backstop in which a claw-like catch hook projects into the inside of the syringe barrel and there engages in a sawtooth-shaped edge of the plunger rod. The catch hook interacts with the sawteeth on the plunger rod in the manner of a pawl mechanism, preventing drawing-back of the plunger stopper.

WO 92/08507 discloses a syringe in which a plate-shaped finger support is fitted onto the proximal end of the syringe barrel. The finger support has a sleeve-like continuation which fits precisely into the syringe barrel and there locks together with a bead at the proximal end of the syringe barrel. The sleeve-like continuation forms a backstop for the plunger stopper, since it narrows the clear internal diameter of the syringe barrel at the proximal end.

Further prior art backstops are disclosed by EP 0 738 517 B1 or EP 0 764 450 B1. In these cases too, the back-stop is combined with a plate-shaped finger support. The finger support is designed such that it can be pushed from the side onto the proximal end of a syringe barrel. A projecting edge narrows the clear internal diameter of the syringe barrel and thus prevents inadvertent removal of the plunger stopper.

From WO 99/55402 it is known to form claw-like catch hooks on the plunger rod itself. With the plunger rod inserted, the catch hooks are inclined towards the proximal end of the syringe barrel and, together with a projection provided in the syringe barrel, prevent removal of the plunger rod.

Further backstops are known from U.S. Pat. No. 5,250,030, DE 44 34 644 C2, DE 199 29 325 A1, and JP 2001-327600 A, inter alia.

Finally, U.S. Pat. No. 4,946,441 describes some sort of a backstop, but one which primarily serves to prevent unauthorized re-use of the syringe after the latter has been used for the first time. Claw-like catch hooks are there a component part of a collar which, at the proximal end of the syringe barrel, is screwed onto an external thread formed there. When it is being screwed on, the catch hooks are pressed radially inwards until they make contact with the plunger rod and prevent withdrawal of the plunger rod from the syringe.

Of the numerous proposals for backstops, there are many that require a special design of the syringe barrel and/or of the plunger rod. All these solutions cannot therefore be applied to “conventional” syringes and are accordingly expensive. Only a few solutions, for instance the backstops from EP 0 738 517 B1, can be used in combination with conventional syringe barrels and plunger rods, so that a conventional syringe can be provided optionally and also subsequently with a backstop.

### SUMMARY OF THE INVENTION

It is an object of the present invention to provide an alternative backstop for a syringe, which backstop can be used in a large number of syringes, particularly in syringes having conventional glass barrels.

It is also an object of the invention to provide a syringe comprising such a backstop.

Accordingly, there is provided a backstop comprising a securing element for securing the backstop at the proximal end of a syringe barrel, and comprising at least two claw-like catch hooks to be arranged outside the syringe barrel at mutually offset circumferential positions, the catch hooks being arranged on a plate-shaped finger support which is designed to be arranged at the proximal end of the syringe barrel.

Moreover, there is provided a syringe comprising a syringe barrel having a proximal end and a distal end, comprising a plunger stopper arranged in the syringe barrel, and comprising a backstop which secures the plunger stopper against inadvertent withdrawal from the syringe barrel, the backstop having at least two claw-like catch hooks arranged outside the syringe barrel at mutually offset circumferential positions, the catch hooks being arranged on a plate-shaped finger support which sits at the proximal end of the syringe barrel.

The novel backstop follows the known approach of seeking to prevent inadvertent removal of the plunger stopper with the aid of claw-like catch hooks. In contrast to the known arrangements, however, the catch hooks here are arranged on a plate-shaped finger support. It is thus possible to use the novel backstop on conventional syringes too. A special configuration of the proximal flange of the syringe is neither essential nor required, as will be shown below with reference to preferred illustrative embodiments.

The novel backstop differs in concept from all previously known backstops for conventional syringe barrels, since it uses claw-like catch hooks located outside the syringe barrel. An advantage is that the novel backstop permits simple removal of the plunger stopper when such removal is deliberately wanted, simply by bending open or detaching one of the catch hooks. Since such a manipulation leaves visible traces, however, the plunger stopper cannot then be introduced back into the syringe barrel without this being noticed. This applies in particular when the backstop as a whole is secured in an irreversible manner on the syringe barrel, as is provided for in a preferred embodiment. By contrast, in the known backstops, deliberate removal of the plunger stopper is either not possible at all or is possible in a way that leaves no visible traces, so that there is a danger of certain manipulations of the syringe remaining unnoticed. None of the previously known backstops offers simple access to the plunger stopper and protects against undetected manipulations.

In a preferred embodiment, the catch hooks are arranged in a freely accessible manner on the finger support.

Freely accessible in this context means that the catch hooks are immediately apparent to the person using the syringe, and said person can reach at least one of the catch hooks so as to bend it open or break it off. This embodiment is therefore particularly suitable for achieving the aforementioned advantages.

In a further embodiment, the catch hooks are inclined with their free ends towards the distal end of the syringe barrel.

This embodiment considerably increases the force by which the backstop is held in relation to the plunger stopper. Accordingly, the danger of the plunger stopper being inadvertently withdrawn from the syringe barrel is further reduced. Moreover, the risk of undetected manipulation of the backstop is also reduced still further.

In a further embodiment, the finger support is a separate structural part which is secured at the proximal end of the syringe barrel.

As an alternative to this, it is in principle also possible for the finger support with the catch hooks to be formed integrally at the proximal end of the syringe barrel. By contrast, the preferred embodiment has the advantage that the novel backstop can be used optionally, and in particular in conventional syringe barrels with a round flange. A further advantage lies in the fact that the novel backstop of this embodiment can easily be made of a material different than that of the syringe barrel. In this way, in particular a backstop made of plastic that withstands high retaining forces can be used on syringe barrels made of glass.

In a further embodiment, the finger support is connected to a wall which has the shape of a cylinder shell and which engages with a precise fit around the proximal end of the syringe barrel.

The wall in the shape of a cylinder shell (that is to say a sleeve-like continuation) permits very stable securing of the novel backstop on conventional syringe barrels. This embodiment is thus particularly well suited for achieving the object set out above.

In a further embodiment, the wall has a lateral opening allowing it to be pushed from the side onto the syringe barrel.

In this embodiment, the novel backstop can be integrated particularly easily and efficiently into existing production processes. Using the novel backstop on conventional syringe barrels is facilitated still further by this.

In a further embodiment, the finger support is secured in a non-releasable manner at the proximal end of the syringe barrel. In a preferred illustrative embodiment, a retainer ring engages in a groove formed on the wall.

This embodiment improves the protection against manipulation and thus makes advantageous use of the possibilities already discussed above. The use of a retainer ring in the manner described permits particularly efficient and economical assembly.

In a further embodiment, the catch hooks are connected integrally to the finger support.

By means of this embodiment, the production costs of the novel syringe and of the novel backstop can be further minimized. In particular, it is possible in this embodiment to produce the catch hooks and the finger support in a single injection moulding step. The associated reduction in the number of parts also permits straightforward assembly of the syringe.

In a further embodiment, the free ends of the catch hooks finish above a terminating plane defined by the proximal end of the syringe barrel.

This embodiment has the advantage that the clear internal diameter of the syringe barrel is not made smaller by the novel backstop. If so desired, the whole internal diameter of the syringe barrel is thus available for receiving the medicament. In addition, this embodiment facilitates deliberate manipulation of the catch hooks.

In a further embodiment, the free ends of the catch hooks end on or outside a circular line corresponding to the clear internal diameter of the syringe barrel.

This embodiment also helps effectively prevent removal of the plunger stopper, without reducing the internal volume of the syringe barrel. Even when the free ends of the catch hooks finish outside the stated circular line, there is effective protection against removal of the plunger stopper, because the plunger stoppers in the syringe barrel are generally compressed in order to achieve good sealing on the inside walls of the syringe barrel. As soon as the plunger stopper leaves the syringe barrel, it expands and thus comes to rest on the catch hooks. This preferred embodiment has the advantage that, upon actuation of the syringe, the plunger rod does not rest on the catch hooks, i.e. the syringe can be actuated unaffected by the backstop. More precise dosing can be achieved in this way.

It will be appreciated that the aforementioned features and the features still to be explained below can be used not just in the respectively stated combination, but also in other combinations or singly, without departing from the scope of the present invention.

### BRIEF DESCRIPTION OF THE DRAWING

Illustrative embodiments of the invention are shown in the drawing and are explained in greater detail in the description below. In the drawing:

FIG. 1 shows an illustrative embodiment of the novel syringe in a side view,

FIG. 2 shows the syringe barrel and the backstop of the illustrative embodiment from FIG. 1 in a perspective view,

FIG. 3 shows the syringe barrel and the backstop according to FIG. 2 in a cross-sectional view, and

FIG. 4 shows the syringe from FIG. 1 without the syringe barrel, in order to illustrate the function of the novel backstop.

### DESCRIPTION OF PREFERRED EMBODIMENTS

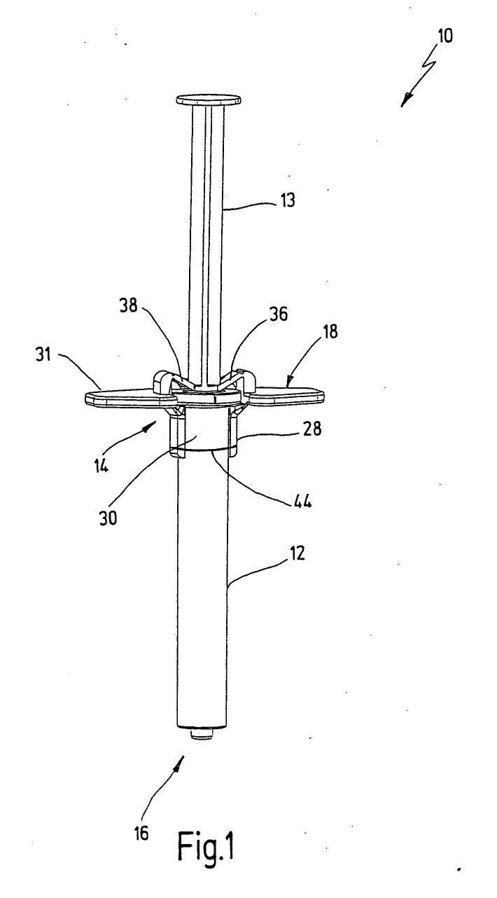

In FIG. 1, an illustrative embodiment of the novel syringe is indicated overall by reference number 10.

The syringe 10 comprises, in a manner known per se, a syringe barrel 12 and a plunger rod 13 which is inserted at the proximal end 14 of the syringe barrel 12. The distal end 16 of the syringe barrel 12 has a connector, for example a Luer lock connector, for applying a needle (not shown here).

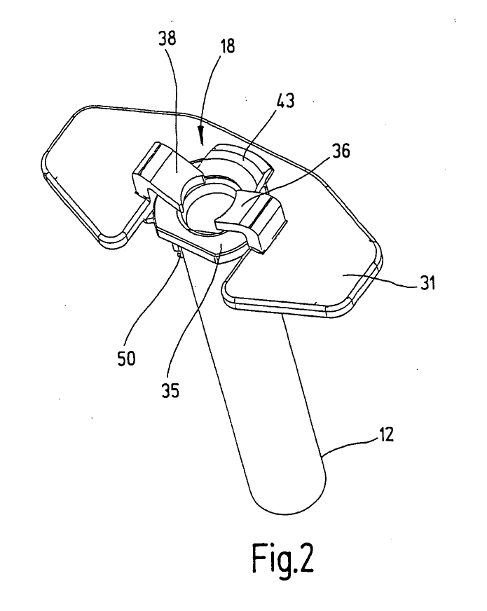

In the further course of the description, reference is made additionally to FIGS. 2 to 4, in which the same reference numbers indicate the same elements.

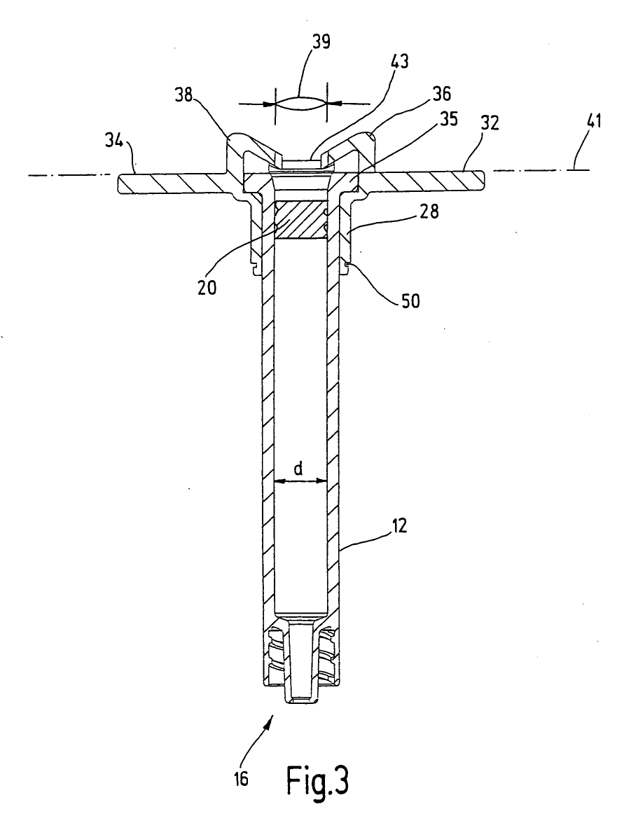

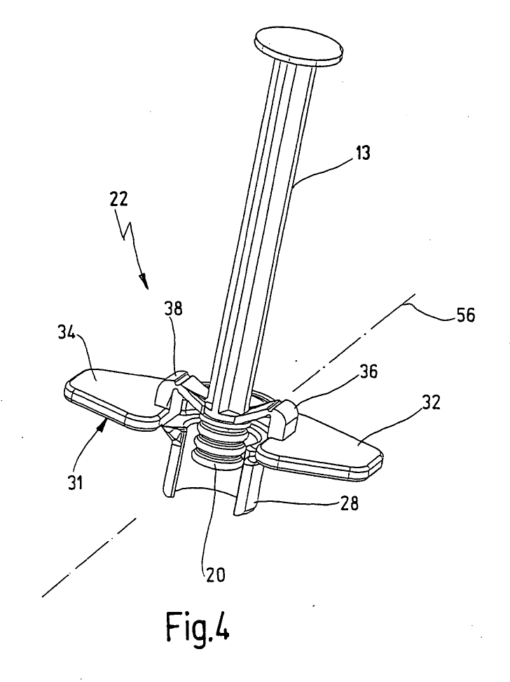

An illustrative embodiment of the novel backstop is indicated by reference number 18. The backstop 18 serves to prevent inadvertent removal of the plunger stopper 20 (see FIG. 3 and FIG. 4) from the syringe barrel 12. Together with the plunger stopper 20, the backstop 18 forms a proximal closure element making it possible to supply the syringe 10 to the end user in the pre-filled state. It goes without saying that the distal end 16 in this case is also sealed off by a closure element, for example by a so-called tip cap or by a needle with needle guard cap.

In the illustrative embodiment shown, the backstop 18 has a wall 28 which has the shape of a cylinder shell and which engages with a precise fit around the proximal end of the syringe barrel 12. The wall 28 has an opening area 30 making it possible to push the backstop 18 from the side onto the proximal end 14 of the syringe barrel 12.

The backstop 18 also has a finger support 31 with two plate-shaped areas 32, 34 which extend transversely with respect to the wall 28 on two opposite sides of the syringe barrel 12. The plate areas 32, 34 end flush with the proximal end of the syringe barrel 12. In other words, the two plate areas 32, 34 engage flush around the proximal end flange 35 of the syringe barrel 12, while the wall 28 of cylinder shell shape bears on the syringe barrel 12 underneath the proximal end flange 35.

Laterally from the syringe barrel 12, a claw-like catch hook 36, 38 is arranged respectively on the plate areas 32, 34. In the presently preferred embodiment shown, the catch hooks 36, 38, the finger support 31 with plate areas 32, 34, and the wall 28 are designed integrally with one another. However, in a deviation from this configuration, the backstop 18 could in principle also comprise a number of parts joined together.

The catch hooks 36, 38 are each approximately L-shaped, a short branch of the L protruding vertically upwards from the respective plate area 32, 34, while a longer branch points radially inwards and is inclined towards the distal end 16 of the syringe barrel 12. The free ends of the catch hooks 36, 38 thus point radially towards one another. In the illustrative embodiment shown, they are located exactly on or slightly outside of a circular line 39 whose diameter corresponds to the clear internal diameter d of the syringe barrel 12. In other illustrative embodiments, however, the free ends of the catch hooks 36, 38 can also protrude into the projected circular line 39.

Moreover, in this illustrative embodiment, the free ends of the catch hooks 36, 38 lie above a terminating plane 41 defined by the proximal end 14 of the syringe barrel 12. In a deviation from this configuration, however, it is in principle also possible for the free ends of the catch hooks 36, 38 to end below the plane 41 and thus protrude into the syringe barrel 12.

Reference number 43 (see FIG. 2) indicates an edge piece which is formed integrally with the finger support 31 of the backstop 18. The edge piece 43 is located on the outer circumference of the syringe barrel 12, approximately centrally between the two catch hooks 36, 38. Its dimensions are chosen such that it lies on the proximal end flange 35 of the syringe barrel 12 without protruding into the clear internal diameter of the syringe barrel 12. The edge piece 43 thus secures the backstop 18 against slipping through to the distal end 16 of the syringe barrel 12.

Reference number 44 in FIG. 1 indicates a retainer ring which runs in a groove 50 formed on the lower (distal) end of the wall 28. The ring 44 prevents the backstop 18 from being drawn off laterally from the syringe barrel 12, so that deliberate manipulations are prevented or at least made noticeable. In a particularly advantageous manner, the ring 44 can be a component part of a tamper-evident closure which in its entirety forms the subject of a parallel patent application (filed on the same date and claiming priority from German patent application DE 10 2004 009 918.9) assigned to the present Assignee, which is incorporated here by reference. In other illustrative embodiments, however, the ring 44 can in principle also be omitted, or the backstop 18 is secured irreversibly on the syringe barrel 12 in some other way, for example by adhesive bonding, shrinking or welding.

FIG. 4 illustrates the principle functioning of the backstop 18. By virtue of the arrangement that has been described, the free ends of the catch hooks 36, 38 can turn about an axis 56 which, for the sake of clarity, is shown only for the catch hook 36. The axis -56 lies above the plane 41 and outside the clear internal diameter d of the syringe barrel 12. If the plunger stopper 20 is withdrawn from the syringe barrel 12, it arrives between the two free ends of the catch hooks 36, 38 and comes to rest there. Further withdrawal of the plunger stopper 20 has the effect that the free ends execute a rotation movement about the corresponding axis 56. The clear distance between the two ends is reduced, and the plunger stopper 20 is therefore clamped more securely. Because of the geometric dimensions of the catch hooks 36, 38, complete removal of the plunger stopper 20 is not possible without destroying said catch hooks.

In the illustrative embodiment shown in the figures, the backstop 18 with the finger support 31 and with the catch hooks 36, 38 is designed in such a way that it can be fitted on conventional syringe barrels 12 having a round flange. Alternatively to this, however, it is also possible to design the backstop 18 in such a way that it can be used in syringe barrels which have another flange. In addition, it is in principle also possible to apply the basic principle of the present backstop 18 in syringe barrels which already have their own finger support 31. In this case too, corresponding catch hooks 36, 38 can be arranged above the finger support 31, either integrally with the latter or via a supplementary assembly part. Moreover, the invention is not limited to illustrative embodiments with two catch hooks. Three or more catch hooks pointing radially towards one another can also be used.

1. A syringe comprising a syringe barrel having a proximal end and a distal end, comprising
a plunger stopper arranged in the syringe barrel,
a backstop which secures the plunger stopper against inadvertent withdrawal from the syringe barrel, and
a plate-shaped finger support at the proximal end of the syringe barrel,
the backstop having at least two claw-like catch hooks arranged on the plate-shaped finger support and outside the syringe barrel at mutually offset circumferential positions.

2. The syringe of claim 1, wherein the catch hooks are arranged in a freely accessible manner on the finger support.

3. The syringe of claim 1, wherein the catch hooks comprise free ends which are inclined towards the distal end of the syringe barrel.

4. The syringe of claim 1, wherein the finger support is a separate structural part which is secured at the proximal end of the syringe barrel.

5. The syringe of claim 1, wherein the finger support is secured in a non-releasable manner at the proximal end of the syringe barrel.

6. The syringe of claim 1, wherein the finger support comprises a wall having the shape of a cylinder shell and engaging with a precise fit around the proximal end of the syringe barrel.

7. The syringe of claim 6, wherein the wall has a lateral opening allowing it to be pushed from the side onto the syringe barrel.

8. The syringe of claim 6, wherein a groove is formed on the wall, the finger support being non-releasably secured at the proximal end of the syringe barrel by means of a retainer which engages in the groove.

9. The syringe of claim 1, wherein the catch hooks are integrally connected to the finger support.

10. The syringe of claim 1, wherein the proximal end of the syringe barrel defines a terminating plane, and the catch hooks comprise free ends finishing above the terminating plane.

11. The syringe of claim 1, wherein the syringe barrel has a clear internal diameter, and the catch hooks comprise free ends ending on or outside a circular line corresponding to the clear internal diameter.

12. A backstop for a syringe having a syringe barrel with a proximal end, the backstop comprising
a plate-shaped finger support designed to be arranged at the proximal end of the syringe barrel,
a securing element for securing the finger support at the proximal end, and
at least two claw-like catch hooks arranged on the plate-shaped finger support such that the claw-like catch hooks come to lie outside the syringe barrel at mutually offset circumferential positions, when the backstop is secured to the syringe barrel.

13. The backstop of claim 12, wherein the catch hooks are arranged in a freely accessible manner on the finger support, when the backstop is secured to the syringe barrel.

14. The backstop of claim 12, wherein the catch hooks comprise free ends which are inclined towards the distal end of the syringe barrel, when the backstop is secured to the syringe barrel.

15. The backstop of claim 12, wherein the securing element comprises a wall having the shape of a cylinder shell and being designed to engage with a precise fit around the proximal end of the syringe barrel.

16. The backstop of claim 15, wherein the wall has a lateral opening allowing it to be pushed from the side onto the syringe barrel.

17. The backstop of claim 16, wherein a groove is formed on the wall for receiving a retainer adapted to prevent removal of the backstop from the syringe barrel once it is pushed onto the syringe barrel.

18. The backstop of claim 12, wherein the catch hooks are integrally connected to the finger support.

19. The backstop of claim 12, wherein the proximal end of the syringe barrel defines a terminating plane, the catch hooks comprising free ends designed to finish above the terminating plane.

20. The backstop of claim 12, wherein the syringe barrel has a clear internal diameter, the catch hooks comprising free ends designed to end on or outside a circular line defined by the clear internal diameter.
